(19) United States
(12) Patent Application Publication
(10) Pub. No.: US 2020/0273559 A1
(43) Pub. Date: Aug. 27, 2020

(54) SYSTEM ARCHITECTURE AND METHODS FOR ANALYZING HEALTH DATA ACROSS GEOGRAPHIC REGIONS BY PRIORITY USING A DECENTRALIZED COMPUTING PLATFORM

(71) Applicant: HeartFlow, Inc., Redwood City, CA (US)

(72) Inventors: Razik YOUSFI, Redwood City, CA (US); Leo GRADY, Redwood City, CA (US)

(73) Assignee: HeartFlow, Inc.

(21) Appl. No.: 16/797,738

(22) Filed: Feb. 21, 2020

Related U.S. Application Data
(60) Provisional application No. 62/809,139, filed on Feb. 22, 2019.

(51) Int. Cl. G16H 30/20; G16H 30/40; G06T 17/20; G06T 19/20; G16H 15/00

(52) U.S. Cl. CPC G16H 30/20; G16H 30/40; G16H 15/00; G06T 19/20; G06T 17/20

(57) ABSTRACT

Systems and methods are disclosed for analyzing health data over a decentralized cloud-computing platform. One method for analyzing health data over a decentralized cloud-computing platform includes: receiving a unique case file containing one or more anonymous DICOM object(s) for analysis; setting a priority level for the unique case file based on priorities associated with the one or more anonymous DICOM object(s); uncompressing and validating at least one of the one or more anonymous DICOM object(s); and transmitting an analysis of at least one of the uncompressed and validated anonymous DICOM object(s), the analysis having been completed according to the priority level of the unique case file.

### RELATED APPLICATION(S)

This application claims priority to U.S. Provisional Application No. 62/809,139 filed Feb. 22, 2019, the entire disclosure of which is hereby incorporated herein by reference in its entirety.

### INTRODUCTION

Hospitals generate and store a significant amount of health data records relating to patients. Traditionally, analysis of health data (e.g., medical records, X-rays, CT scans, MRI, etc.) is performed by medical professionals at the hospitals. However, a hospital may not be equipped to perform predictive health data analysis involving cutting edge technologies. In these situations, health data may need to be analyzed by third party vendors or services.

Increasingly, various aspects of health data analysis are performed outside of the hospital. When the health data are analyzed using one or more third party processing system(s) outside the hospital, there may be various factors to consider for transferring and analyzing the health data out of a hospital, e.g., file size, type, routing, file adequacy (e.g., image quality), Application User Interfaces (API) to be used to facilitate the selection of files to transfer, and privacy.

Additionally, the third party processing system(s) may require a robust platform architecture in order to efficiently analyze and organize the data from many different hospitals from different regions (or countries). For example, a particular set of health data analysis, storage, and management capabilities may exist only within a certain region that is different from the hospital region. Hospitals outside of that selected region may not have access to the analysis services, since patient privacy regulations may dictate that patient health data cannot be transferred to the selected region.

A desire thus exists for a decentralized health data platform that creates a secure, scalable, robust, elastic infrastructure for the end-to-end automated ingestion, analyzing and processing of cross-border health data, and reporting of the analysis. A desire further exists for the decentralized health data platform to provide guided analyst workflow in order to enhance the data analysis and reporting to the hospital(s). The foregoing general description and the following detailed description are exemplary and explanatory only and are not restrictive of the disclosure. Various embodiments of the present disclosure relate to systems and methods that preserve patient privacy while efficiently analyzing, managing, and storing health data generated in different geographical areas, according to one embodiment.

### SUMMARY

According to certain aspects of the present disclosure, systems and methods are disclosed for managing cross-border health data over a decentralized cloud-computing platform.

A method of analyzing health data over a decentralized cloud-computing platform, the method includes: receiving a unique case file containing one or more anonymous DICOM object(s) for analysis; setting a priority level for the unique case file based on priorities associated with the one or more anonymous DICOM object(s); uncompressing and validating at least one of the one or more anonymous DICOM object(s); and transmitting an analysis of at least one of the uncompressed and validated anonymous DICOM object(s), the analysis having been completed according to the priority level of the unique case file.

In accordance with another embodiment, a system for analyzing health data over a decentralized cloud-computing platform comprises: a data storage device storing instructions for analyzing health data over a decentralized cloud-computing platform; and a processor configured for: receiving a unique case file containing one or more anonymous DICOM object(s) for analysis; setting a priority level for the unique case file based on priorities associated with the one or more anonymous DICOM object(s); uncompressing and validating at least one of the one or more anonymous DICOM object(s); and transmitting an analysis of at least one of the uncompressed and validated anonymous DICOM object(s), the analysis having been completed according to the priority level of the unique case file.

In accordance with another embodiment, a non-transitory computer readable medium for use on a computer system containing computer-executable programming instructions for performing a method of analyzing health data over a decentralized cloud-computing platform, the method comprising: receiving a unique case file containing one or more anonymous DICOM object(s) for analysis; setting a priority level for the unique case file based on priorities associated with the one or more anonymous DICOM object(s); uncompressing and validating at least one of the one or more anonymous DICOM object(s); and transmitting an analysis of at least one of the uncompressed and validated anonymous DICOM object(s), the analysis having been completed according to the priority level of the unique case file.

Additional objects and advantages of the disclosed embodiments will be set forth in part in the description that follows, and in part will be apparent from the description, or may be learned by practice of the disclosed embodiments. The objects and advantages of the disclosed embodiments will be realized and attained by means of the elements and combinations particularly pointed out in the appended claims.

It is to be understood that both the foregoing general description and the following detailed description are exemplary and explanatory only and are not restrictive of the disclosed embodiments, as claimed.

### BRIEF DESCRIPTION OF THE DRAWINGS

FIG. 1A depicts an exemplary block diagram of a decentralized health data management system, according to an exemplary embodiment of the present disclosure.

FIG. 1B depicts an exemplary schematic diagram of a health data IO system of FIG. 1A, according to an exemplary embodiment of the present disclosure.

FIG. 1C depicts an exemplary schematic diagram of a health data analysis system of FIG. 1A, according to an exemplary embodiment of the present disclosure.

FIG. 2 illustrates is a detailed block-diagram of the health data analysis system of FIG. 1C, according to an exemplary embodiment of the present disclosure.

FIG. 3 is a block diagram of an exemplary method of balancing network load in health data IO system 101 of FIG. 1A, according to an exemplary embodiment of the present disclosure.

FIG. 4 is a sequence diagram of a method of receiving, analyzing, and reporting health data, according to an exemplary embodiment of the present disclosure.

FIG. 5 is a flow diagram of an exemplary method of managing the order of case processing, according to an exemplary embodiment of the present disclosure.

FIG. 6 is a flow diagram of a method of analyzing cross-border health data using decentralized cloud-computing platform, according to an exemplary embodiment of the present disclosure.

FIG. 7 is a flow diagram of an exemplary method of post-processing and reporting result files from a health data analysis system, according to an exemplary embodiment of the present disclosure.

### DESCRIPTION OF THE EMBODIMENTS

Reference will now be made in detail to the exemplary embodiments of the disclosure, examples of which are illustrated in the accompanying drawings. Wherever possible, the same reference numbers will be used throughout the drawings to refer to the same or like parts.

As used herein, the term “exemplary” is used in the sense of “example,” rather than “ideal.” Moreover, the terms “a” and “an” herein do not denote a limitation of quantity, but rather denote the presence of one or more of the referenced items. For the purposes of the disclosure, “patient” may refer to any individual or person for whom diagnosis or treatment analysis (e.g., data analysis) is being performed, or any individual or person associated with the diagnosis or treatment analysis of one or more individuals.

The term, “health data” may include medical data (e.g., Digital Imaging and Communications in Medicine (DICOM) objects) collected for a patient in one practice, as well as health data collected for the patient by any health care providers, labs, specialists, clinicians, etc. Health data may be associated with a particular patient, where the patient is identifiable via patient privacy information. Patient privacy information may be any information that links health data to a particular individual. Patient privacy information may be defined objectively (as information that ties an individual to his/her health data) and/or bylaws and regulations. Various regions may include different definitions of what information that constitutes patient privacy information.

“Region” may refer to any environment with borders defined by regulations protecting patient privacy. In one embodiment, “region” may refer to a known geographic region governed by a set of regulations that protect patient privacy/personal data. For example, “region” may refer to a state (e.g., California), a country (e.g., the United States, Japan, Canada, etc.), or a group of countries (e.g., Europe) operating under a single set of patient privacy laws. For the United States, patient privacy information may include protected health information (PHI). For the United States and various other regions, patient privacy information may include personal health information, patient identification information, etc. (e.g., CT, MRI, ultrasound, etc.). Various systems and methods for protecting patient health information while transmitting health data are disclosed, for example, in U.S. Non-Provisional application Ser. No. 15/635,127 filed Jun. 27, 2017 and entitled “Systems and Methods for Modifying and Reacting Health Data Across Geographic Regions,” which is hereby incorporated herein by reference in its entirety.

Alternately or in addition, “region” may refer to a given physical facility, a given entity's computing systems, or a given cloud platform. In such cases, various regions between which health data is redacted may nevertheless exist within a single country, state, or province. For example, a region may include a hospital that may remove patient privacy information from collected health data prior to transferring the health data to another region (e.g., a cloud platform) for data analysis. The hospital and the cloud platform may exist in the same geographically defined region, e.g., the United States.

The present disclosure includes a system and method for managing health data over a decentralized cloud computing platform. In one embodiment, the health data may be pre-processed, analyzed, and post-processed for reporting using one or more cloud-based web service(s). The health data management system may receive health data from one or more hospital(s), perform automatic health data ingestion, prepare health data for analysis, analyze/transform health data in preparation for analysis, select or identify files to transfer for analysis, time the transfer of a health data file for analysis, determine whether health data files are related, monitor the progress or status of health data analysis, determine completed analyses of health data, and otherwise facilitate the interactions between a hospital or DICOM modality and various cloud-based data analysis web service(s). In one embodiment, the health data may be in the form of DICOM objects.

In one embodiment, a health data management system may transfer health data (e.g. DICOM objects) from customer sites (e.g., hospitals, clinics, etc.) to cloud computing web service(s). For example, the system may receive health data directly from CT scanners, CT workstations, or a picture archiving and communication system (PACS) using DICOM protocol and securely transfer the received health data to cloud services. The cloud computing services may include data analysis services that may derive one or more diagnostic metrics or patient recommendations based on the received health data. The system may further retrieve a completed analysis of the transferred health data, aggregate analysis results from one or more cloud services, generate reports, and/or push reports to hospital systems.

In one embodiment, the health data may include patient-specific image data. In one scenario, image data may include data regarding the geometry of the patient's heart, e.g., at least a portion of the patient's aorta, a proximal portion of the main coronary arteries (and the branches extending therefrom) connected to the aorta, and the myocardium. In one embodiment, the patient-specific image data may be in the form of DICOM files. The patient-specific anatomical data may be obtained noninvasively, e.g., using a noninvasive imaging method. For example, CCTA is an imaging method in which a user may operate a computer tomography (CT) scanner to view and create images of structures, e.g., the myocardium, the aorta, the main coronary arteries, and other blood vessels connected thereto. The CCTA data may be time-varying, e.g., to show changes in vessel shape over a cardiac cycle. CCTA may be used to produce an image of the patient's heart.

Alternatively, other noninvasive imaging methods, including magnetic resonance imaging (MRI) or ultrasound (US), or invasive imaging methods, such as digital subtraction angiography (DSA), may be used to produce images of structures of a patient's anatomy. The imaging methods may involve injecting the patient intravenously with a contrast agent to enable identification of the structures of the anatomy. The health data involving images (e.g., provided by CCTA, MRI, etc.) may be provided by a third-party vendor, such as a radiology lab or a cardiologist, by the patient's physician, etc.

Other patient-specific anatomical data may also be determined from the patient noninvasively. For example, physiological data such as the patient's blood pressure, baseline heart rate, height, weight, hematocrit, stroke volume, etc., may be measured. The blood pressure may be the blood pressure in the patient's brachial artery (e.g., using a pressure cuff), such as the maximum (systolic) and minimum (diastolic) pressures. Exemplary health data may thus include data collected from imaging modalities, wearables, blood pressure cuffs, thermometers, fitness trackers, glucometers, heart rate monitors, etc.

In one embodiment, preparing files for analysis and retrieving analyzed data may involve preserving patient privacy. For example, file preparation may include removing patient privacy information from the files. In one such case, health data files may automatically include patient privacy information when the files are generated. Exemplary patient privacy information may include protected health information (PHI) or a patient privacy data equivalent to PHI. In one scenario, file preparation may include stripping health data files of patient privacy information before the health data is transferred from a hospital, to a health data analysis system for analysis. In one embodiment, result reports comprising analyzed health data may be generated, absent patient privacy information. The reports may be pushed to a hospital/hospital interface, where the report may be coupled to corresponding patient privacy information. In this way, data analysis may be performed in a remote region, without transmitting patient privacy information to the remote region.

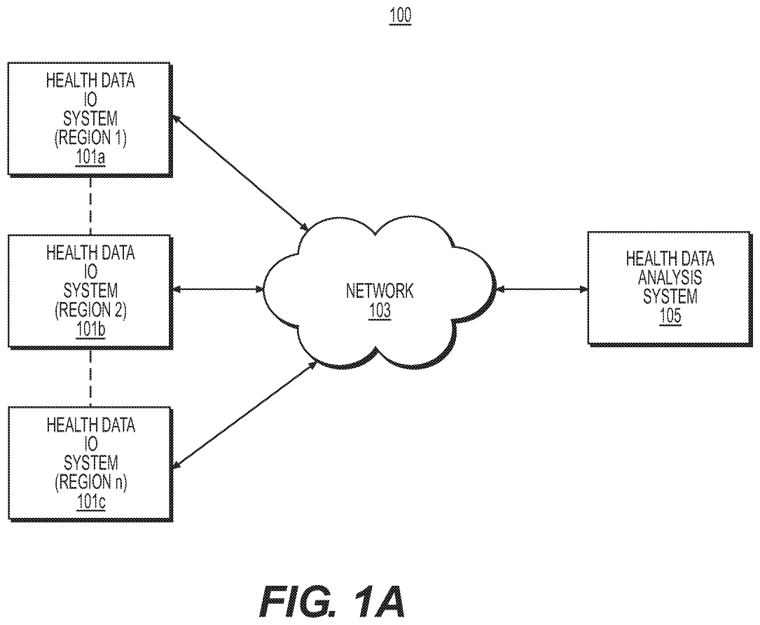

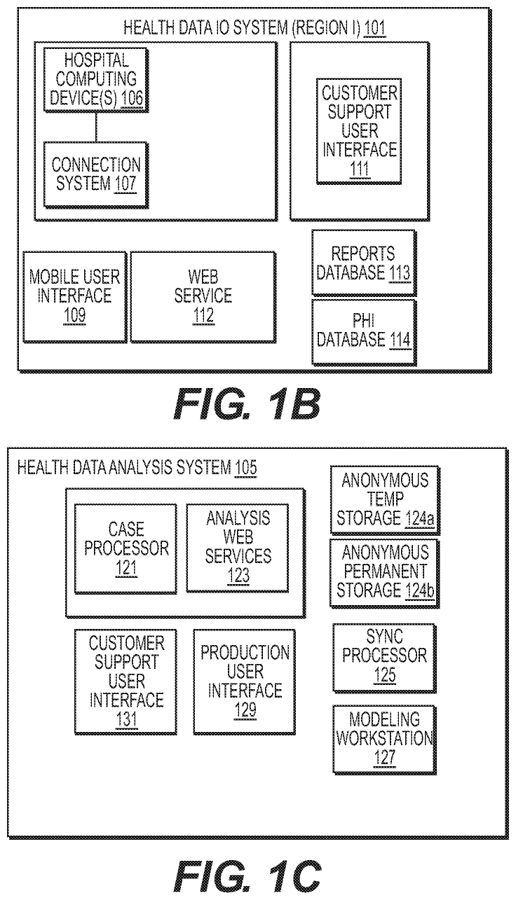

FIG. 1A is a block diagram of decentralized health data management system 100 for managing and analyzing health data over a cloud platform, according to an exemplary embodiment of present disclosure. The health data infrastructure may comprise one or more health data input-output (10) system(s) 101 (101a-101n) and health data analysis system 105, where each of the health data input-output systems may be customers of health data analysis system 105. For example, the health data analysis system 105 may provide a service comprising generating analyzed data from health data (e.g., diagnostic metrics, treatment simulations, treatment optimization or recommendations, etc.). The health data input-output system 101 may then send health data to the health data analysis system 105 to have the service performed on the provided health data.

In one embodiment, each of the one or more health data IO system(s) 101 may be located in a region different from the location of the health data analysis system 105. One or more health data system(s) 101 may convey health data as input to a health data analysis system 105 over a wireless network 103. Alternately, the health data may be conveyed to the health data analysis system 105 over a wired connection. The health data IO system 101 may also receive reports of analyzed health data output from health data analysis system 105 over a wireless network 103.

In one embodiment, the health data IO system(s) 101 may each represent different pre-determined regions, where patient privacy information may not be transferred freely from one region to another. In one embodiment, the boundary of a region may be defined such that patient privacy information may pass freely within a boundary of the region, and patient privacy information may not be transferred freely once past the boundary of the region. For example, the boundary of the region may be based on government regulations surrounding transfer of patient privacy information. In such cases, regions may refer to physical geographical regions (e.g., Europe, Japan, and the U.S.), where the government of each region may dictate different rules on the transfer of patient privacy information. Alternately or in addition, the health data IO system 101 may represent regions where boundaries dictated by health codes, insurance policies, hospital policies, and/or local/state/provincial regulations. For example, the health data IO system 101 may represent various facilities (e.g., distinct hospitals, hospital networks, lab facilities, patient groups, etc.). An exemplary demarcation between a health data IO system 101 and a health data analysis system 105 may include an embodiment where health data is generated at a health data IO system 101, and there are technical and legal barriers to transfer of the health data to the health data analysis system 105 for analysis.

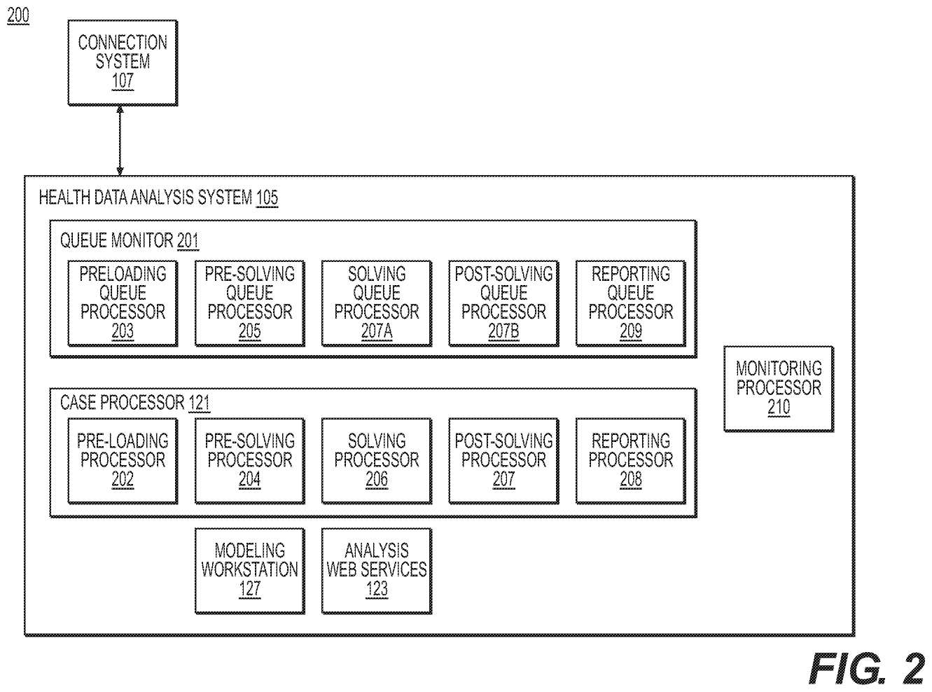

In one embodiment, each health data IO system 101 may be associated with a pre-set priority level. The priority level may dictate the order in which health data is submitted for review (e.g., as described in FIG. 2), transmitted for analysis (e.g., by health data analysis system 105), as well as the order in which reports of analyzed data may be integrated or delivered (e.g., from the health data analysis system 105 to the health data IO system 101). One exemplary scenario may include health data IO system 101a being associated with a “high priority” priority level and a health data IO system 101b being associated with a “low priority” priority level. In such a case, even if a first set of health data is generated by health data IO system 101a at the same time that second set of health data is generated by health data IO system 101b, the first set of data may be transferred to health data analysis system 105 and health data analysis system 105 may convey reports of the analyzed data back to health data IO system 101a before the second set of health data from health data IO system 101b is transferred to or analyzed by health data analysis system 105.

Alternately or in addition, each health data IO system 101 may be associated with multiple priority levels. For example, health data IO system 101a may include multiple priority settings organized, for example, by task items (e.g., “actions”), priority status, (e.g., “low priority”, “high priority”, “emergency status”), or standard processes (e.g., “check-up update”, “monitoring update”, etc.). The priority settings may be pre-set upon configuration of a data transmission channel between the health data IO system 101a and health data analysis system 105. Priority may refer, not only, to a priority status for the data transmission, but also an order in which data processing is to be performed. Alternately or in addition, the priority settings may be associated with the device generating the health data, dictated by clinicians/health care providers upon generation of the health data, dictated by the analysis requested, etc. In other words, the priority of a case file of DICOM objects may be based on an entity generating one or more DICOM object(s) (associated with the one or more anonymous DICOM objects) and/or an entity analyzing the one or more anonymous DICOM objects of the case file.

The health data may be analyzed by health data analysis system 105 in an order according to the priority level of the health data set within each health data IO system 101 (e.g., relative to other sets of health data produced by the health data IO system 101), and according to the priority level of the health data IO system (e.g., relative to other health data IO systems). In one embodiment, the type of health data may also affect the priority status. For example, if a particular set of health data is found to be associated with a stored or previously-created set of health data, the particular set of health data may automatically be deemed “prioritized” or “high priority,” regardless of the priority level of the stored or previously-created set of health data. In other words, priority settings may be comprised of and impacted by various factors, and there may be several layers of priorities in health data management system 100.

In one embodiment, users may dictate priority settings and define how various factors impact or change priority upon initializing the health data management system 100, or upon initializing the communication between a health data IO system 101 and the health data analysis system 105. Alternately or in addition, health data analysis system 105 may pre-define at least one or more priority settings, so that some priority settings are consistent or used by default across all health data IO system 101 customers of health data analysis system 105.

In addition to priority, other properties may be associated with health data during uploading of the health data from each health data IO system 101 to the health data analysis system 105. Properties associated with the health data may include, for example, imaging modality, referring physician, experimental test code, lab code, billing code, patient health plan, insurance plan, etc. In one embodiment, a property determined during the uploading of the health data may prompt one or more specific algorithms to be run by the health data analysis system 105, either directly in analyzing the data, or in an auxiliary way (e.g., in preparing a report of the data or the timing in which the data is received or reported).

It should be appreciated that health data IO system 101 and health data analysis system 105 may include any type or combination of computing systems, e.g., handheld devices, personal computers, servers, clustered computing machines, and/or cloud computing systems. In one embodiment, health data IO system 101 and health data analysis system 105 may comprise an assembly of hardware, including a memory, a central processing unit (“CPU”), and/or a user interface. The memory may include any type of RAM or ROM embodied in a physical storage medium, such as magnetic storage including floppy disk, hard disk, or magnetic tape; semiconductor storage such as solid state disk (SSD) or flash memory; optical disc storage; or magneto-optical disc storage. The CPU may include one or more processors for processing data according to instructions stored in the memory.

FIG. 1B is a detailed schematic block-diagram of an exemplary health data IO system 101 of FIG. 1A, according to an exemplary embodiment of present disclosure. The health data IO system 101 may be comprised of a hospital interface including one or more hospital computing device(s) 106, a data transport system (“connection system”) 107, and a mobile user interface 109. One embodiment of the health data IO system 101 may also include a web service 112, a report(s) database 113, and a patient health information database (e.g., PHI database 114). A customer support user interface 111 may be in communication with the web service 112. Web service 112 may be comprised of a cloud service. The health data IO system 101 may further include a metadata database. These components of the health data IO system 101 may be located in the same region or country as one or more hospitals.

The hospital computing device(s) 106 may include any type of electronic device configured to collect, send, and/or receive data, including websites, models, medical data, health records, multimedia content, etc. Exemplary hospital computing devices may include medical devices, e.g., medical imaging devices, medical monitors, etc. Hospital computing device(s) 106 may also include one or more mobile devices, smartphones, personal digital assistants (“PDA”), tablet computers, or any other kind of touchscreen-enabled device, a personal computer, a laptop, and/or server. In one embodiment, each of the hospital computing device(s) 106 may have a web browser and/or mobile browser installed for receiving and displaying electronic content received from one or more of web servers affiliated with health data IO system 101. The hospital computing device(s) 106 may also include client devices that may have an operating system configured to execute a web or mobile browser, and any type of application, e.g., a mobile application. In one embodiment, various hospital computing device(s) 106 may be configured with network adapters to communicate data or analyzed reports. Alternatively, or additionally, various hospital computing device(s) 106 may be configured to transmit data or receive analyzed data over a local network connection.

In an example embodiment, the hospital device may be a computerized tomography (CT) scan workstation, CT scanner, or a picture archiving and communication system (PACS) system installed within the hospital. In one embodiment, the hospital computing device(s) 106 may upload health data to the connection system 107 over a wireless network. The connection system 107 may serve as a connection between the health data IO system 101/hospital computing device(s) 106 and health data analysis system 105 (e.g., by pushing DICOM objects generated by the hospital computing device(s) 106 to a remote health data analysis system 105).

In one embodiment, the health data may be uploaded to the connection system 107 from a hospital computing device 106. For example, a DICOM study (comprising a CT scan) may be pushed from a DICOM modality hospital computing device 106 to connection system 107. Connection system 107 may create a new case file corresponding to the DICOM study. In addition, connection system 107 may prompt the creation of a new case account or file at the health data analysis system 105. In an alternative embodiment, the connection system 107 may query hospital computing device(s) such as (CT workstation, CT scanner, and PACS) and pull the health data (e.g., DICOM objects) from such computing device(s). In one embodiment, a hospital computing device may be in communication with a web server that saves DICOM objects. Connection system 107 may push or pull DICOM objects from this web server.

In one embodiment, connection system 107 may communicate with the web server at regular time intervals to pull any DICOM objects available via the server. Alternately or in addition, a processor associated with generating DICOM objects may be in local communication with the connection system 107, wherein the connection system 107 may be prompted to pull DICOM objects when DICOM objects are generated or stored.

Alternately or in addition, a pull mechanism may further be initiated by requests for any tasks, e.g., for analysis of DICOM objects. In one embodiment, DICOM objects may be associated with various data, e.g., priority of the analysis, type of case study to be conducted, patient scan/images, patient privacy information, etc. This data may be metadata or tags included in a DICOM object file. Such components of the DICOM objects may affect the order in which files are sent for analysis. In one instance, DICOM objects may be stored until data analysis is requested. A pull mechanism used to transfer cases to health data analysis system 105 may be governed by case priority level(s) or case type(s) that are associated with each DICOM object. For example, health data may be labeled with a “type,” which may pull data for analysis, based on the rate or order dictated by a data type or priority setting. Exemplary types may include the following: emergency, prioritized, retrospective, urgent, low priority, test, research, commercial, standard, demo, onboarding, monitoring, prospective, clinical study. Types may be stacked, for instance, “urgent” cases may be further sorted into “emergency” or “prioritized,” where “emergency” may indicate high priority due to a patient's health condition being in a critical state and “prioritized” may indicate need for expedited analysis for a reason other than a patient's immediate health condition. Settings of case types may be set up at hospital computing device(s) 106, a mobile user interface 109, and/or by a connection system 107. The pull mechanisms may be configured to minimize overlapping data pulls or overloading of the health data IO system 101, connection system 107, and health data analysis system 105.

Alternately or in addition, a hospital user or a clinician may upload health data to the connection system 107 using a mobile user interface 109. The mobile user interface 109 may be the customer-facing web interface (e.g., to hospital users). The mobile user interface 109 may present a login interface to one or more users, perform password management, display a case list, search, display case details, and display a user profile. The mobile user interface 109 may also be used to access reports containing analyzed data associated with a patient. The mobile user interface 109 may comprise a mobile application.

In one embodiment, data generated by hospital computing device(s) 106 may be associated with patient privacy information, and patient privacy information of a DICOM object may be available to a web service 112, but not available or accessible from entities outside of the health data IO system 101. For example, connection system 107 may extract patient privacy information (e.g., PHI data) from a DICOM object and store the patient privacy information such that it is available to a web service 112 within the health data IO system 101 (e.g., PHI database 114). For example, connection system 107 may split DICOM tags containing patient privacy information from DICOM images. The DICOM tags containing patient privacy information may be replaced with a predefined string. As an alternative embodiment, a web service 112 may receive DICOM objects from connection system 107, and extract patient privacy information from the DICOM objects. The web service 112 may further store the patient privacy information in a database (e.g., PHI database 114). In an example embodiment, a region-specific customer service provider may have access to patient privacy information (PHI) and patient result report(s) using a customer support user interface 111.

In one embodiment, connection system 107 may create a case file for data analysis using an application programming interface (API) deployed in a health data IO system 101 (e.g., a region associated with a hospital). Patient privacy information stripped from DICOM file(s) of the case may be submitted to the API, which may store the submitted patient privacy information to a patient privacy information database (e.g., PHI database 114). Once a case is processed (e.g., data of the DICOM file(s) is analyzed by health data analysis system 105), case/analysis completion may trigger a reporting process deployed in the health data IO system 101, meaning a report may be generated using the analyzed data and patient privacy information stored in the patient privacy information database (e.g., PHI database 114). In one embodiment, the patient privacy information may be retrieved using the API.

Patient privacy information may always remain in the region in which health data for a case (e.g., a DICOM object) was generated or the region from which the health data was pushed. Patient privacy information may be stored in storage service buckets and/or patient privacy information databases in each of the regions (e.g., health data IO system 101 of FIG. 1A).

In one embodiment, all of the patient privacy information may be encrypted in-transit and at-rest. In one embodiment, storage service buckets for patient privacy information may have server-side encryption enabled, such that encryption may occur each time a new object is stored. Patient privacy information databases (e.g., PHI database 114) may also offer encryption services, where patient privacy information may be encrypted upon storage, and each time the patient privacy information is accessed.

In one embodiment, patient privacy information may not be accessed outside of a region in which it is stored. For example, a user who submitted a case in Japan may be able to access various web services while he/she is in another region (e.g., Europe), but the user may not be able to access patient privacy information of health data management system 100 while he/she is in the other region.

In an example embodiment, the web service 112 may receive reports of analyzed data from the health data analysis system 105. For example, the web service 112 may determine that completed reports are available in reports database 113. In one embodiment, completed reports may be associated with a unique case identifier. In one embodiment, the web service 112 may retrieve patient privacy information from PHI database 114 associated with the retrieved case results using unique case identifier. The web service 112 may further integrate PHI data with the result reports and/or store the reports in reports database 113. The web service 112 may further store reports in the reports database 113, either along with patient privacy information, or with a unique case identifier. In one embodiment, the health data IO system 101 may further include a metadata database. The metadata database may include metadata associated with each report or set of health data. Metadata may include data associated with health data known at creation of the data, e.g., time at which the data was created or imaged, insurance plan, billing information, etc. Metadata may also include data created in the process of analysis of the health data (e.g., by health data analysis system 105), e.g., notes or markings from physicians or reviewers, pins from health data pre-processing (for instance, if anatomical models are constructed from the health data), etc.

In one embodiment, the web service 112 may include a reporting queue monitor, which may apply the order or priority in which reports are accessed or made available for usage (e.g., by clinicians, health care personnel, patients, etc.). The priority may be set by user(s) or web services (e.g., web service 112). In one embodiment, the reporting queue may manage cases in order of case priority (as dictated by the case “type”). The health data IO system 101 may further include a customer support user interface 111. The customer support user interface 111 may be accessible to a customer support provider (e.g., customer support personnel). In one embodiment, patient privacy information and/or patient reports may be accessed via a Virtual Private Network (VP N) configured for that usage by the customer support provider. For example, customer support personnel may have the ability to log into each region individually by leveraging the VPN service. The VPN service may extend access to patient privacy information of a specific region to the customer support provider, even when the customer support provider is not accessing the patient privacy information from the specific region at which health data was generated.

FIG. 1C is a schematic block-diagram of health data analysis system 105, according to an exemplary embodiment of present disclosure. The health data analysis system 105 may be comprised of a case processor 121, one or more analysis web service(s) 123, anonymous temporary storage 124a, anonymous permanent storage 124b, sync processor 125, and modeling workstation 127. The health data analysis system 105 may further provide user interfaces to analysts or customer support personnel to review and/or access data from one or more analysis web service(s) 123. In one embodiment, the user interfaces may include a production user interface 129 and/or a customer support user interface 131.

The health data analysis system 105 may serve as one or more cloud platform(s) for analyzing anonymous health data (e.g., via connection system 107) from at least one cross-border region. More specifically, the analysis web service(s) 123 may compute results and generate reports (e.g., 3D model, PDF, etc.) to present analyses and results to a patient, clinician, or user. For example, one or more analysis web service(s) 123 may compute blood flow within coronary arteries using received image data from cardiac CT scans. Various embodiments of diagnostic analyses using image reconstructions and anatomic models are disclosed, for example, in U.S. Pat. No. 8,315,812 issued Nov. 20, 2012, entitled “Method and System for Patient-Specific Modeling of Blood Flow,” which is incorporated by reference in its entirety. The one or more analysis web service(s) 123 may provide a report of the results of the analysis performed by analysis web service(s) 123. For the analysis described above, a report may include an interactive 3D model of the coronary arteries showing the computed Fractional Flow Reserve (FFR) values and/or a PDF report. An exemplary report may include a surface model, graph, chart, color-coding, indicators, interactive displays, alerts, signals, or any known graphical objects. Various embodiments of graphical representations and displays are disclosed, for example, in U.S. Pat. No. 8,706,457 issued Apr. 22, 2014, entitled “Method and System for Providing Information from a Patient-Specific Model of Blood Flow,” which is incorporated by reference in its entirety.

In an example embodiment, one or more processor(s) associated with the case processor 121 may generate one or more intermediate results from the received DICOM objects. In an example embodiment, sync processor 125 may synchronize intermediate results from one or more processor(s) and the intermediate results may be accessed for review via modeling workstation 127. In an example embodiment, sync processor 125 may be an application launched from a user interface accessible by people (e.g., technicians or analysts) processing cases. In one embodiment, sync processor 125 may further launch the modeling workstation 127 for each specific case. Sync processor 125 may ensure a proper communication between one or more analysis web services 123 and the modeling workstation 127. In an example embodiment, an analyst may view the intermediate results from case processing, update the results generated by the case processor 121, or submit a request to reanalyze a case via the case processor 121.

In an example embodiment, the health data analysis system 105 may include multiple front ends, including a production user interface 129 and a customer support user interface 131. In an example embodiment, the production user interface 129 may serve as an internal front-end for case analysts and quality control of the analysis web service(s) 123. The production user interface 129 may also serve as a portal to internal processing application(s) for health data analysis system 105. In one scenario, the production user interface 129 may provide an analyst with the capabilities to process cases, manage priorities, and report and review cases before they are delivered to a customer. The production user interface 129 may also facilitate the analyst in validating analysis results, for example, by identifying analysis results that may have relatively low statistical confidence, and prompting the analyst to review (or prioritize the review of) the analysis result. The customer support user interface 131 may serve as an internal front-end for site creation, user management, and dashboarding. The customer support user interface 131 may be used by support staff to monitor and manage sites and institutions, e.g., by way of connection system 107 (shown in FIG. 1B). In one embodiment, the support user interface 131 may also manage data quarantines, e.g., for product testing, development, or other tool development functions. For example, cases may have one or more tags, e.g., quarantined, restricted, or general. These tags may be generated when cases are created. The tags may be used or updated after each case report is completed, to see whether the raw or analyzed data can be used for future development/analysis. Customer support user interface 131 may also generate reports of cases processed over a given or selected period of time, e.g., for internal use or accounting to run a report of case(s) generated for free, by contract, for payment, for research, etc.

FIG. 2 depicts a detailed block diagram 200 of the health data analysis system 105 of FIG. 1C, according to an exemplary embodiment of present disclosure. In an example embodiment, connection system 107 may extract patient privacy information from DICOM objects of a case file so that the case file is anonymous. The connection system 107 may then send the anonymous case file to health data analysis system 105 using HTTP OR HTTPS secure Hypertext Transfer Protocol (HTTPS). In one embodiment, the connection system 107 may create a unique case identifier for the anonymous case file, so that the anonymous case file may be tracked using the unique case identifier, rather than using patient privacy information.

In an example embodiment, case processor 121 may process the DICOM objects of each case using a preloading processor 202, (a modeling workstation 127), a pre-solving processor 204, a solving processor 206, a post-solving processor 207, and a reporting processor 208. In an example embodiment, a case may be processed through the preloading processor 202, a pre-solving processor 204, a solving processor 206, a post-solving processor 207, and a reporting processor 208 according to the set priorities of the case. In an example embodiment, a hospital clinician or administrator may enter a priority setting for one or more DICOM object source CT scanners, CT workstations, or PACS using a web interface. In an alternative embodiment, the priority may be set automatically according to the location of a hospital device in a hospital. The priority may be sent to the case processor 121 using a stateless protocol. In each stage or processing, the cases with may be queued according to priority and high priority cases may be and processed prior to cases of lower priority. Alternately or in addition, case priorities may be edited at any stage of the process.

In one embodiment, a preloading processor 202 may prepare or verify that received DICOM files are ready for analysis. For example, preloading processor 202 may uncompress (or decompress) DICOM files received from connection system 107. In one embodiment, connection system 107 may also have the capabilities to uncompress DICOM files, but preloading processor 202 may provide a more secure environment for uncompressing DICOM files, relative to connection system 107. Preloading processor 202 may also perform a verification of the quality of the DICOM files as meeting a minimum threshold necessary for the requested analysis to be performed by health data analysis system 105. For example, a blood flow analysis may entail CT image quality meeting a minimum, predetermined threshold value in order for the resultant blood flow metrics to be clinically accurate. A plaque detection analysis may have a lower predetermined threshold value of image quality for the results to be useful to a patient. In one embodiment, connection system 107 may automatically reject received health data. As another embodiment, preloading processor 202 may also have the capability to automatically reject received health data. For example, preloading processor 202 may reject a case if the health data is missing DICOM images, has incorrect image spacing, incorrect z-positioning, etc. If the DICOM object fails to meet the quality assessment of preloading processor 202, connection system 107 may receive a notification that the DICOM file is rejected for analysis, or that analysis cannot be performed using the uploaded DICOM object. In one embodiment, the health data IO system 101 may be prompted to generate or upload a new or updated DICOM file for analysis. For example, a mobile user interface 109 may be prompted to display a case as “returned.” As another example, a “returned” case may include an imaging quality rejection report, including the reasons for the health data being rejected for analysis. If new health data is sent that is accepted, a new case entity may be created, and any associated data may be copied to the new case/duplicate case.

In one embodiment, if the uncompressed DICOM files have an accuracy or image quality level that meets a predetermined threshold accuracy or image quality level, the uncompressed DICOM files may pass directly to pre-solving processor 204. If the files to not meet the predetermined accuracy or image quality threshold level(s), the file may be transferred to modeling workstation 127 for manual processing, e.g., using a workstation to create a mesh (anatomical model). In some embodiments, the mesh model created by modeling workstation 127 may then be transferred to preloading processor 202.

In one embodiment, pre-solving processor 204 may perform preliminary computations to convert the image files into a form to be used to the requested analysis. For example, a blood flow analysis of health data analysis system 105 may be based on a 3-dimensional (“3D”) model of a patient's vasculature. Pre-solving processor 204 may convert raw DICOM image files into a 3D volumetric model for the blood flow analysis. In one embodiment, pre-solving processor 204 may further generate a mesh of tetrahedral elements and/or ascertain initial boundary conditions to be used for an analysis. In one embodiment, pre-solving processor 204 may be in communication with a modeling workstation 127 to render a model for validation by an analyst of the health data analysis system 105. If the requested analysis includes a blood flow simulation, pre-solving processor 204 may determine initial boundary conditions along various locations of a 3D model of a patient's vasculature.

In one embodiment, solving processor 206 may launch analysis of prepared models rendered by pre-solving processor 204. For example, solving processor 206 may produce a blood flow simulation through a 3D model produced by pre-solving processor 204. In one embodiment, solving processor 206 may interface with analysis web service(s) 123 to perform an analysis. In one embodiment, solving processor 206 may include a post-solving processor, which may aggregate analyses performed by solving processor 206 and package the aggregated analyses for the reporting processor 208. For example, the post-solving processor may identify previous analyses or stored analyses related to the current analysis performed by solving processor 206. The analyses may be related via the type of analysis performed (e.g., blood flow simulation or perfusion simulation), analyses linked by association with a patient or patient demographic, etc. (Although the data may be anonymized (e.g., dissociated from patient privacy information), various sets of data may still be identified as being associated with the same individual by tracking or hashing a unique ID associated with the anonymized data.) The analyses may include images and models of a user-specific blood flow and possible treatment outcomes. In one embodiment, reporting processor 208 may generate views, displays, pins, PDFs, and/or interactive interfaces and models that may constitute a final report of the assessments performed by the solving processor (and post-solving processor) 206. Reporting processor 208 may allow review and revisions to be made in the analyses generated by solving processor 206.

In one embodiment, each component of the case processor may have corresponding queue monitor(s) 201 (e.g., preloading queue processor 203 corresponding to preloading processor 202, a pre-solving queue processor 205 corresponding to pre-solving processor 204, a solving queue processor 207a and a post-solving queue processor 207b corresponding to solving processor 206, and a reporting queue processor 209 corresponding to reporting processor 208). The queue monitor(s) 201 may advance cases through each of their corresponding components. For example, the preloading queue processor 203 may dictate the progress of each image data set through preloading processor 202. In one embodiment, an API may be used to capture the status of each case as it advances through states of analysis.

Each of the queue monitor(s) may periodically poll for indications of cases being available from their respective components of the case processor and download the related data from storage. For example, preloading queue processor 203 may detect that a new DICOM data set has been uploaded. Preloading queue processor 203 may then download the new data set and prompt preloading processor 202 to verify that the data set meets image quality requirements. Preloading queue processor 203 may detect from preloading processor 202 that the data set meets image quality standards, and initiate action from pre-solving processor 204 to generate a geometrical or volumetric model based on the data set. Likewise, solving queue processor 207a may detect that a geometrical or volumetric model is available and fit for analysis, and thus prompt solving processor 206 (and analysis web services 123) to initiate the analysis of simulating blood flow through the geometrical or volumetric model. In one embodiment, post-solving queue processor 207b may detect the completion of an analysis by solving processor 206. The post-solving queue processor 207b may then prompt post-solving processor 207 to validate the results. In some embodiments, this validation may include presenting the results to a modeling workstation 127 for an analyst to confirm the analysis outcome. The reporting queue processor 209 may initiate reporting processor 208's creation of a report from validated results.

In one embodiment, the queue monitor 201 may further process one or more received cases (e.g., DICOM objects) according to one or more priorities set for the received case. For example, priorities may be set by the health data IO system 101 and/or priorities may be set in association with the analyses requested for the health data. The queue monitor(s) may manage each stage of data analysis, depending on the priority assigned to each case. For example, a low priority case that is at the pre-solving processor stage may remain at the pre-solving processor stage while a subsequent high priority case is routed to the solving processor stage, ahead of the low priority case. The queue monitor(s) further monitor the bandwidth of the analysis web services 123 so that cases are processed at a steady pace.

The monitoring processor 210 in FIG. 2 may be responsible for monitoring the status of the case processing and analysis. The monitoring processor 210 may monitor state of the case (e.g., SUCCESS, FAIL, etc.) at each level as the case is processed through the preloading processor 202, a pre-solving processor 204, a solving processor 206, and a reporting processor 208. In an example embodiment, the monitoring processor 210 may be invoked or disabled by one or more authorized users using remote commands. The monitoring processor 210 may execute the commands under the context of a user specifically created with limited rights just for the purpose of executing remote commands. The monitoring processor 210 may notify one or more API(s) associated with front-end user interface(s) about status of the case (e.g., DICOM object analysis). The monitoring processor 210 may generate one or more automatic processing event(s) using one or more status messages (e.g., SUCCESS, FAILURE, etc.) generated by the unique case at one or more stages of processing.

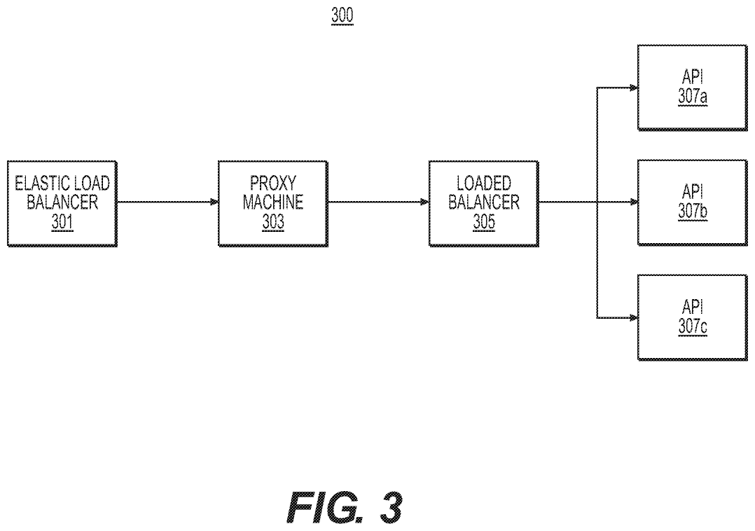

FIG. 3 is an exemplary block diagram 300 of load balancing to bridge the communications between the front ends (including a clinical user interface (e.g., mobile user interface 109 or customer support user interface 111, web service 112, analysis web services 123, or any cloud, local, analysis web services)), databases, and back-ends (e.g., web services or case processors), according to an exemplary embodiment of the present disclosure. In one embodiment, health data management system 100 may horizontally scale its APIs 307 (e.g., API 307a-307c). For example, health data management system 100 may employ an elastic load balancer 301 that may use multiple proxy machines 303 of a lower profile provided behind a load balancer 305 that may distribute the network load across APIs 307 efficiently. Alternately or in addition, health data management system 100 may include advanced machine hardware to handle the load encountered by health data management system 100.

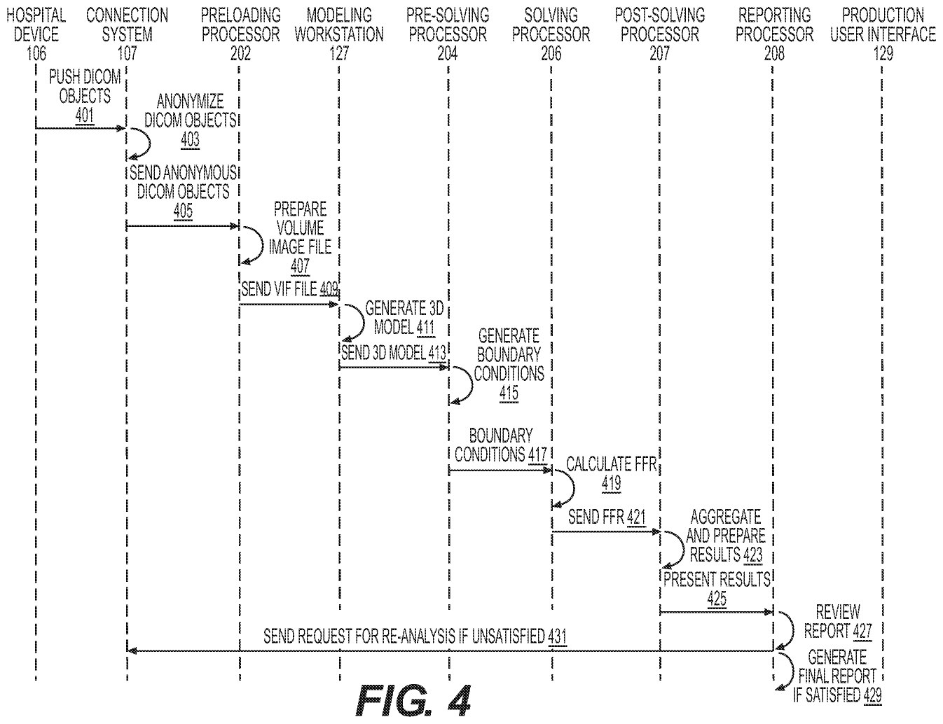

FIG. 4 depicts a sequence diagram of an exemplary method 400 of receiving, analyzing, and reporting health data, according to an exemplary embodiment of the present disclosure. In an example embodiment, a DICOM object may be pushed to the connection system 107 from one or more hospital computing device(s) 106. Once the connection system 107 receives the DICOM object (e.g., step 401), the connection system 107 may create a new case file associated with the DICOM object. This case file may be monitored throughout progression of the analysis of the DICOM object. The case file may also be monitored through analysis, until a final report is available.

In one embodiment, connection system 107 may extract patient privacy information (e.g., PHI data) from the DICOM objects (e.g., step 403). In doing so, the DICOM objects may be anonymized. In one embodiment, the patient privacy information may be sent to a region-specific cloud platform (e.g., web service 112) using a service that may permit stateless operations. For example, the patient privacy information may be sent to the region-specific cloud platform via a Representational State Transfer (REST) Application Programming Interface (API). The region-specific cloud platform may store the patient privacy information in a local relational database (e.g., a database structured to recognize relations among stored items of information). PHI database 114 is one example of such a local relational database. Various systems and methods for anonymizing DICOM objects are disclosed, for example, in U.S. Non-Provisional application Ser. No. 15/635,127 filed Jun. 27, 2017 and entitled “Systems and Methods for Modifying and Reacting Health Data Across Geographic Regions,” which is hereby incorporated herein by reference in its entirety.

In one embodiment, step 405 may include transmitting anonymized DICOM objects to a preloading processor (e.g., preloading processor 202). In one embodiment, either the connection system 107 and/or the preloading processor 202 may uncompress and validate the DICOM objects according to pre-configured validation criteria. The validation criteria may be dependent on the analysis to be performed on the DICOM object.

In one embodiment, the preloading processor 202 may further prepare volume image files from the received DICOM objects (e.g., step 407). In one embodiment, the preloading processor 202 may further generate a compressed archive file that may pertain to one or more volume image files. In one embodiment, the compressed archive file may contain information corresponding to the volume image file(s). The information may pertain to the analysis of the volume image file(s). For example, the compressed archive file may include a user log, a task state, a type of processing that may be performed using the volume image file, etc. A user log may include a history of the file, including previous processing performed on the volume image file, actions performed on a model by user(s), etc. A task state may include the state of each task for generating a volume image file, as well as the time spent in each task. Exemplary tasks may include the following: identify aorta, identify vessel landmarks, identify myocardial tissue, identify centerlines, label vasculature, review lumen geometry, finalize model, etc. The type of processing that may be performed using the volume image file may be dictated by the health data IO system 101, a particular user of the health data IO system 101, or the health data analysis system 105, or dictated by the type of data received. For example, step 407 may include correcting misregistrations in received data or having pre-set operations, for example, processing files more if the received data includes only one object phase. In one embodiment, manipulations to the validated DICOM objects or volume image file prepare the validated DICOM objects for analysis (e.g., by the health data analysis system 105 or cleaning by modeling workstation 127). In one embodiment, processed images may be sent to the modeling workstation 127 for an additional check on image quality. This additional check may lead to a rejection of image validation. In one embodiment, connection system 107 may perform a first level of validation of received images, and preloading processor 202 may perform an image-related validation (e.g., checking if any anatomical spacing or thickness is erroneous in received data). In one embodiment, modeling workstation 127 may review image data further, e.g., for whirring or image artifacts. In one embodiment, the correspondence or linkage between a compressed archive file and a volume image file may be maintained using a checksum. The checksum may be computed by the preloading processor 202, connection system 107, an API, or any component of the system 100. In one embodiment, volume image files of the preloading processor 202 may be presented to an analyst, for instance, via modeling workstation 127. For example, step 409 may include presenting one or more volume image files using a user interface of modeling workstation 127. In one embodiment, the modeling workstation 127 may be used to generate a patient-specific anatomic model (e.g., step 411). The model may include a three-dimensional (3D) geometry of a patient's vasculature (e.g., a 3D mesh model of a patient's coronary artery tree). In one embodiment, the patient-specific 3D model may be generated from volume image file(s) using one or more automated image analysis algorithms.

In one embodiment, the modeling workstation 127 may present the 3D model to an analyst for review (e.g., via production user interface 129). The modeling workstation 127 may present the 3D model in a series of validation screens, and prompt an analyst to approve or reject various portions of the model. In one embodiment, the modeling workstation 127 may receive input from the analyst and store the analyst's response in the compressed archive file (e.g., as part of the user log). The input may include case notes from the analyst. In an example embodiment, the modeling workstation 127 may prompt re-uploading of DICOM objects if a model is not approved. For example, one or more hospital computing device 106 may receive a prompt (e.g., via mobile user interface 109) to generate additional DICOM object(s). In an alternative embodiment, the modeling workstation 127 may convey a request to preloading processor 202 to regenerate volume image files. In an example embodiment, the modeling workstation 127 may convert the 3D model and case notes, e.g., into an Extensible Markup Language (XML) file. The output from modeling workstation 127 may be used in an analysis (e.g., to simulate blood flow and produce one or more blood flow metrics).

Once a 3D model is approved, a pre-solving processor (e.g., pre-solving processor 204) may perform one or more operations to further prepare for analysis by health data analysis system 105. In one embodiment, the pre-solving processor may receive an approved 3D model (e.g., step 413) and generate boundary conditions for a blood flow simulation through the 3D model (e.g., step 415). In one embodiment, the pre-solving processor may use one or more analytics algorithms to generate a mesh of tetrahedral elements from the 3D model (which may be in an XML file). The pre-solving processor 204 may calculate a set of initial boundary conditions to be used as input to a blood flow simulation computations. The pre-solving processor 204 may then convey the initial boundary conditions to the solving processor (e.g., step 417).

In one embodiment, a solving processor (e.g., solving processor 206) may perform an analysis (e.g., while employing analysis web service(s) 123). For example, the solving processor 206 may perform a blood flow simulation using the three dimensional (3D) mesh model of tetrahedral elements and the initial boundary conditions (e.g., step 419). In one scenario, the solving processor 206 may calculate one or more blood flow metrics based on the blood flow simulation. The blood flow metrics may include fractional flow reserve (FFR), blood pressure, blood flow velocity, perfusion, plaque vulnerability metric(s), etc.

In one embodiment, analysis results of the solving processor 206 may be received and compiled by a post-solving processor (e.g., post-solving processor 207). For example, solving processor 206 may send calculated blood flow metrics from the blood flow simulation analysis as results to a post-solving processor (e.g., step 421). In one embodiment, a post-solving processor (e.g., post-solving processor 207) may aggregate one or more files generated by the solving processor 206 (e.g., step 423). The post-solving processor 207 may further remove items from analysis files that are not desired in final reports, including notes or pins in the simulation that may not contribute to final results.

Alternately or in addition, a reporting processor (e.g., reporting processor 208) may aggregate the result data and prepare a report for the completed analysis (e.g., step 423). For example, the reporting processor 208 may generate a report of the completed analysis, including initial model view(s) with automated pins pointing areas of interest and PDF files. For example, reporting processor 208 may locate areas of stenosis in modeled vasculature (e.g., based on an analysis comprising blood flow simulation or computation).

In one embodiment, steps 427 and 429 may include presenting the generated reports and pins for review and editing (e.g., via production user interface 129). The annotations may be generated automatically by reporting processor 208. In one embodiment, the 3D model report may be modified, for instance, at the production user interface 129. For instance, the production user interface 129 may prompt an analyst to review the report and adjust the position of one or more pins for the area of relevancy (e.g., main blood vessels) in various model view(s). Additionally, the production user interface 129 may provide the ability to add more pins or notes to the report(s), create relevant view(s), and generate PDF report(s) containing details of the analysis (e.g., the blood flow simulation). The PDF report may be provided to the health data IO system 101 for storage (e.g., at the reports database 113 of FIG. 1B) and access (e.g., via one or more mobile user interfaces 109). Additionally, an analysis may be prompted if an analyst is not satisfied with the results. In an example embodiment, re-triggering analysis may transition a case back to any of the preloading, modeling, pre-solving, solving, post-solving or reporting state(s) (e.g., step 431). In one embodiment, manual intervention (e.g., via modeling workstation 127) may be bypassed if cases of received unprocessed data meet preset requisite accuracy or image quality levels. After going through pre-solving processor 204, solving processor 206, and post-solving processor 207 operations, a report may be delivered to a customer (e.g., by reporting processor 208). Such an embodiment may bypass further review (e.g., of reports or pins in reports by modeling workstation 127).

In an example embodiment, a case monitor (e.g., case processor 121) 429 may notify an API of the health data IO system 101 that a report is completed. In one embodiment, the report may be matched to patient privacy information (e.g., PHI of PHI database 114), and the patient privacy information may be integrated into the report. The report may then be pushed to a requesting hospital workstation, PACS, or an HL7 engine/broker to be accessed by a patient and/or health care professional.

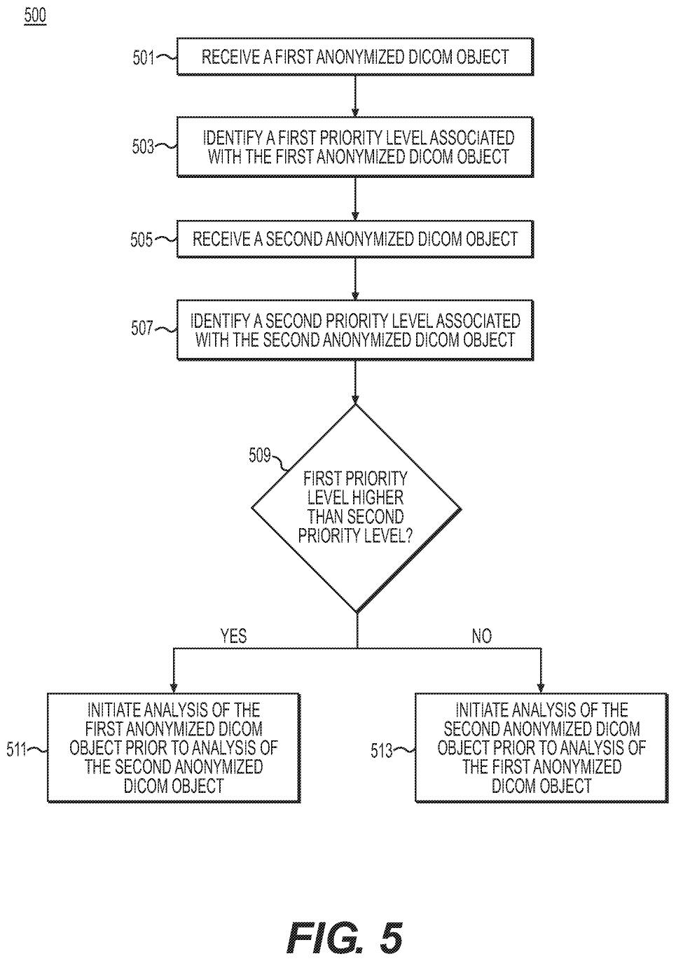

FIG. 5 is a flow diagram of an exemplary method of managing the order of case processing (by the health data analysis system 105), according to an exemplary embodiment of the present disclosure. In one embodiment, steps 501 and 503 may include receiving a first anonymized DICOM object and identifying a priority level associated with the first anonymized DICOM object. An anonymized DICOM object may include one or more image files devoid of patient privacy information. In one embodiment, the priority level may be associated with the health data IO system 101 that generated the DICOM object. For example, various priority level(s) may be configured upon the establishment of communication between the health data IO system 101 and health data analysis system 105. Priority levels may further be set by analysis or health care providers requesting the analysis. For example, health data IO system 101 may prompt a user to set a priority level to be associated with the generated DICOM object.

In one embodiment, steps 505 and 507 may include receiving a second anonymized DICOM object and identifying a priority level associated with the second DICOM object. Step 509 may include determining which case to process first (e.g., at health data analysis system 105). In one embodiment, step 509 may include comparing the priority level associated with the first anonymized DICOM object against the priority level associated with the second anonymized DICOM object. If the priority level of the first anonymized DICOM object is higher than the priority level associated with the second anonymized DICOM object, then analysis may be initiated for the first anonymized DICOM object prior to analysis of the second anonymized DICOM object (e.g., step 511). For example, step 511 may include the queue monitor 201 (e.g., of FIG. 2) queuing the first anonymized DICOM object for processing through the various components of a case processor 121 prior to pushing the second anonymized DICOM object through the progression of steps of the case processor 121. For instance, the first anonymized DICOM object may undergo one of the steps of validation by the preloading processor 202, preparation for analysis by the pre-solving processor 204, analysis by the solving processor 206, or a combination thereof, prior to the second anonymized DICOM object. A report of the analysis based on the first anonymized DICOM object may also be generated and made available to the health data IO system 101 prior to completion of a final report based on the second anonymized DICOM object.

If the priority level of the first anonymized DICOM object is lower than the priority level associated with the second anonymized DICOM object, then analysis may be initiated for the second anonymized DICOM object prior to analysis of the first anonymized DICOM object (e.g., step 513) For example, the second anonymized DICOM object may undergo at least one of processing through the preloading processor 202, pre-solving processor 204, solving processor 206, reporting processor 208, or a combination thereof, prior to the first anonymized DICOM object.

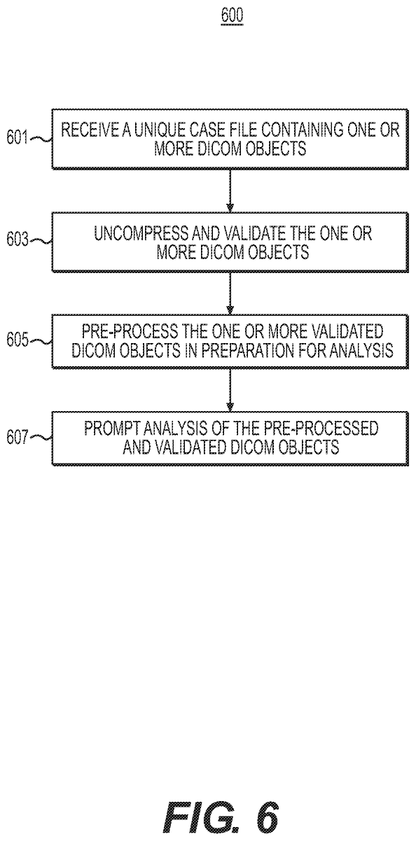

FIG. 6 is a flow diagram of an exemplary method of analyzing cross-border health data efficiently over a cloud-computing platform, according to an exemplary embodiment of the present disclosure. In one embodiment, step 601 may include receiving health data as a unique case file containing one or more DICOM objects. The case file may include one or more priority levels associated with it, where the priority level(s) may dictate the order in which the case goes through analysis at health data analysis system 105. In one embodiment, a unique case identifier may be generated for the received health data (e.g., DICOM objects).

In one embodiment, step 603 may include un-compressing and extracting DICOM images from the received data. In one embodiment, step 603 may include generating, for each unique case, a compressed archive file from extracted DICOM images. The compressed archive file may include a customized file that contains a header file, a one series-select file, a history file (e.g., a user log), a task-states file, a process ID file, a set (n>=1) of folders representing a series of computations that have been processed, etc. The compressed archive file may also include one or more volume image files, depending on the health data received. For example, if health data is generated by a CT scanner, the compressed archive file may include one or more volume image files comprising CT scans.

In one embodiment, step 603 may include validating one or more DICOM object(s) of the received unique case. For example, a DICOM object may be deemed invalid if the DICOM object is missing a DICOM image, if the DICOM image size of a DICOM object is outside of a defined range, or if the DICOM image does not meet defined quality criteria. In one embodiment, step 603 may include setting one or more quality standards associated with each analysis available from the health data analysis system 105 (e.g., via analysis web service(s) 123). For example, if an analysis offered by health data analysis system 105 includes performing a blood flow simulation based on an anatomic model built from DICOM objects, step 603 may include minimum image quality requirements for building anatomic models that would render simulation results that can be clinically used. Step 603 may further include generating confidence parameters based on image quality parameters. For instance, step 603 may include assessing the image quality of received DICOM object(s) and comparing an image quality score of the received DICOM object(s) against pre-determined image quality requirements. Step 603 may further include determining that the DICOM object(s)′ image quality score meets the pre-determined image quality requirements and generating a metadata tag including a confidence score for blood flow simulation results, based on the determining that the DICOM object(s)′ image quality score. Step 603 may further include determining that received DICOM object(s) are invalid. Step 603 may then include generating a prompt to re-take or re-submit health data for the use case. The prompt may be sent directly to a hospital computing device 106, or the prompt may be sent to a user via a user interface.

In one embodiment, step 605 may include pre-processing the DICOM object. For example, step 605 may include determining an analysis to be performed using the received DICOM object; step 605 may further include pre-processing the DICOM object in preparation for the determined analysis. For example, if an analysis includes blood flow simulation, a pre-processing step may include generating an anatomical model based on the DICOM object. The anatomical model may include a 3D mesh model. In one scenario, pre-processing may include extracting DICOM images from the received DICOM object(s) and generating a 3D volumetric model from the extracted DICOM images. The blood flow simulation analysis may then be performed by simulating blood flow through the generated 3D mesh model. Various systems and methods for simulating blood flow are disclosed, for example, in U.S. Pat. No. 8,315,812 filed Jan. 25, 2011 and entitled “Method and System for Patient-Specific Modeling of Blood Flow,” which is hereby incorporated herein by reference in its entirety. In one embodiment, step 605 may include determining pre-processing desired for each analysis available from health data analysis system 105. Step 605 may further include determining uses for results of pre-processing. For example, the generated 3D mesh model may be used for analyses other than blood flow simulation(s). Alternately or in addition, 3D mesh models used for analyses for one metric (e.g., blood pressure) may be used in analyses for a different metric (e.g., plaque vulnerability). In one scenario, step 605 may also include determining the extent of pre-processing desired for a certain analysis. For example, the DICOM object may be used to supplement a 3D mesh model associated with the unique case ID of the case, whereas the DICOM object may be used to generate a new 3D mesh model if the unique case identifier does not already have a model associated with it.

In one embodiment, the 3D mesh model may be in the form of 3D volume files created using representational state transfer (REST) HTTP protocol. In an example embodiment, the volume image files may include a header, binary data, custom headers (e.g., tracking mechanisms or identifiers supported by analysis web service(s) 123), etc. The header may include a series unique identification (UID). The UID may be retrieved from a DICOM object header associated with the case file under analysis.

In one embodiment, each volume image file of image data may correspond to a compressed archive file, storing information regarding a 3D model generated based on the image data of the volume image file. The compressed archive file may further be converted into a file that includes representations of the 3D model that may be both human-readable and machine-readable (e.g., an Extensive Markup Language (XML) file). The file may include a 3D model mesh and other information, including centerlines, vessel boundaries, vessel diameters/radii, tissue boundaries, pins (e.g., markings made by analysts), image quality scores, confidence metrics, etc.

In one embodiment, each of the volume image files may contain a file checksum. The file checksum may be used to correlate series UID with model data (e.g., lumen segmentation and/or myocardial mass), associate a compressed archive file with one or more volume image file(s) (e.g., for longitudinal studies or data tracking), and ensure the one or more volume image file(s) are not corrupted over time. In one embodiment, the checksum may be computed using a raw data part of the volume image file with an off-the-shelf algorithm. The identifier for the checksum algorithm may be contained in the checksum itself. This arrangement may be used to handle various versions or upgrades of the checksum algorithm. In one embodiment, the compressed archive file may include volume image file checksums in order to maintain a consistent link between the compressed archive file and the image data. In one embodiment, various models may be created from the image data, e.g., using different segmentation algorithms, focusing on different portions of the image data, etc.

In one embodiment, step 607 may include prompting analysis of the results of pre-processing. For example, if the pre-processing of step 605 generated a 3D model, step 607 may include prompting an analysis using the 3D model. In one embodiment, step 607 may be performed according to the set priorities of the case file, as per FIG. 5. In one embodiment, step 607 may include sending one or more one or more pre-processed anonymous DICOM objects to analysis web service(s) 123 for analysis. In an example embodiment, a modeling workstation may compute the checksum for a volume image files if a checksum is not already included in transmitted volume image files. A checksum may be used to match a volume image file to a compressed archive file. For example, a volume image file may be named based on its own checksum, thus facilitating correlation with information contained within the compressed archive file. For example, a modeling workstation may retrieve a list of volume image files associated with a given compressed archive file.

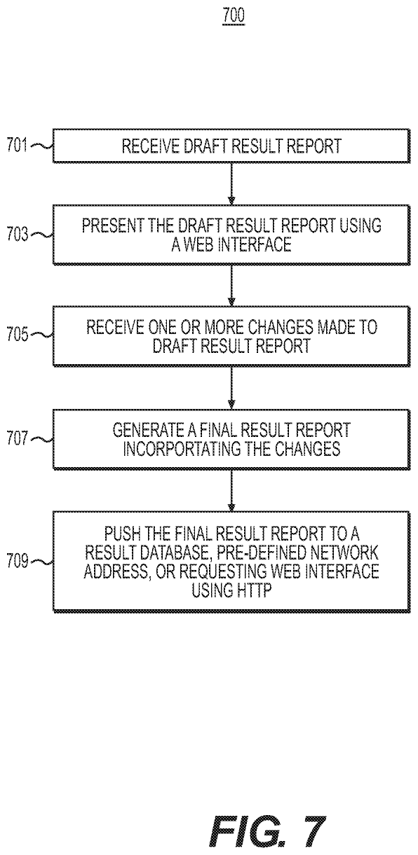

FIG. 7 is a flow diagram of an exemplary method 700 of post-processing result files from health data analysis system 105, according to an exemplary embodiment of the present disclosure. In an example embodiment, step 701 may include receiving a draft result report from the analysis web services 123. In one embodiment, a pre-determined format for presenting a final result report may be predetermined, and any items in the draft result reports that are not part of the final result report may be removed. For instance, analysis web services 123 may return several simulations of stent insertions to treat a stenosis in a patient's vasculature. Multiple simulation results may be removed so that the draft result report shows only the most conservative simulation (e.g., the stent insertion that may be least invasive and produce the highest hemodynamic impact). In one embodiment, step 701 may further include identifying whether there are previous results from analysis web services 123 related to the draft result report, and aggregating the draft result report with the previous results.

In one embodiment, step 703 may include presenting the draft result report/aggregated report to an analyst for review (using an interactive user interface, e.g., the modeling workstation 127 or the production user interface 129 of FIG. 1C). In one embodiment, step 705 may include receiving one or more changes/modifications to the draft result reports, e.g., edits to views of the draft report, pins, simulations, etc. In one embodiment, step 707 may include re-triggering analysis and generation of a draft result report.

In an example embodiment, step 707 may include generating a final result report incorporating one or more changes/modifications provided by the analyst via the user interface. In the example embodiment, the final result report may include a PDF document and/or an interactive 3D model with pins or marks emphasizing relevant areas of the model and solutions from the analysis. For example, the final result report may include a 3D model of a patient's anatomy, blood flow metrics calculated based on the 3D model, comparisons of the hemodynamic impact of various geometric modifications to the 3D model (e.g., from the insertion of a stent or graft, a change in a physiological state, medication, etc.). In one embodiment, the final result report may be stored (e.g., in the anonymous permanent storage 124b of FIG. 1C) for longitudinal studies and/or machine learning purposes for various analyses of the analysis web services 123.

In one embodiment, step 709 may include pushing the final result report to a health data IO system 101 (e.g., via a pre-defined network address of a hospital system or a web interface of the health data IO system 101). In an example embodiment, step 709 may include retrieving the pre-defined network address from one or more DICOM objects used to generate the final result report. In an example embodiment, a user (e.g., hospital technician) may request the result report for a case using a web application (e.g. online portal) and a unique case identifier. The result report may be pushed to the health data IO system 101 in a specific format (e.g., PDF format or 3D model format). The health data IO system 101 may receive the request for result report from the user through HTTP Protocol.

In an alternate embodiment, the health data analysis system 105 may push the result report to the hospital computing system (e.g., PACS or CT workstation) when the result reports are ready. For example, case status may be monitored (e.g., by queue monitors) and results may be pushed to a hospital system or clinical user interface (e.g., of health data IO system 101) once the case is transitioned into COMPLETED state. In an example embodiment, pushing the health data report to at least one of a hospital computing device, hospital electronics records server, and pre-configured destination network address may comprise sending a HL7 standard message to at least one of a hospital computing device, hospital electronics records server, and pre-configured destination network address. In one embodiment, the HL7 standard message may contain the health data report as an encapsulated datatype or URL.

The present disclosure includes a system and method for analyzing, storing, and managing patient's health data in different geographical regions while preserving patient privacy data. The present disclosure further includes a system and method for sending anonymous health data to an analysis cloud platform outside of the region of the hospital and transmitting an analysis result report. Accordingly, data analysis may be provided across regions, while avoiding the transfer of privacy data from one region to another. Any of the described embodiments may be modified, for example, to include variations of data that may be kept within a region. The disclosed systems and methods may be modified to model and assess any range of changes to circulation.

The functions of one or more processors associated with one or more cloud based web services may be provided by a single dedicated processor or by a plurality of processors. Moreover, the processor may include, without limitation, digital signal processor (DSP) hardware, or any other hardware capable of executing software. Program aspects of the technology may be thought of as “products” or “articles of manufacture” typically in the form of executable code and/or associated data that is carried on or embodied in a type of machine readable medium. “Storage” type media include any or all of the tangible memory of the computers, processors or the like, or associated modules thereof, such as various semiconductor memories, tape drives, disk drives and the like, which may provide non-transitory storage at any time for the software programming.

All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A computer-implemented method of analyzing health data over a decentralized cloud-computing platform, the method comprising:
receiving a unique case file containing one or more anonymous DICOM object(s) for analysis;
setting a priority level for the unique case file based on priorities associated with the one or more anonymous DICOM object(s);
uncompressing and validating at least one of the one or more anonymous DICOM object(s); and
transmitting an analysis of at least one of the uncompressed and validated anonymous DICOM object(s), the analysis having been completed according to the priority level of the unique case file.

2. The computer-implemented method of claim 1, wherein the analysis comprises:
generating a volume image file based on at least one of the anonymous DICOM object(s) of the unique case file, the volume image file representing a three dimensional (3D) volume;
generating an archive file including analysis data associated with the volume image file; and
generating a patient-specific model based on the volume image file and the archive file.

3. The computer-implemented method of claim 2, wherein the volume image file includes a file checksum that correlates the volume image file to the archive file.

4. The computer-implemented method of claim 1, further comprising:
monitoring a status of the unique case file; and
advancing analysis of the unique case file based on the status and the priority level of the unique case file.

5. The computer-implemented method of claim 1, further comprising:
setting the priority level for the unique case file based on an entity generating one or more DICOM object(s) associated with the one or more anonymous DICOM objects and/or an entity analyzing the one or more anonymous DICOM objects.

6. The computer-implemented method of claim 1, further comprising:
generating a draft result report based on the received analysis; and
generating an interactive user interface of the draft result report.

7. The computer-implemented method of claim 1, further comprising:
receiving a modification to the draft result report;
generating a final result report based on the modification; and
storing the final result report, a pre-defined identified network address, or a requesting web interface.

8. The computer-implemented method of claim 6, wherein the final result report is pushed to a database, a pre-defined identified network address, or a requesting web interface using secure Hypertext Transfer Protocol (HTTPS).

9. A system for analyzing health data over a decentralized cloud-computing platform, the system comprising:
one or more data storages devices storing instructions of analyzing health data over a decentralized cloud-computing platform; and
one or more processors configured to execute the instructions to perform a method including:
receiving a unique case file containing one or more anonymous DICOM object(s) for analysis;
setting a priority level for the unique case file based on priorities associated with the one or more anonymous DICOM object(s);
uncompressing and validating at least one of the one or more anonymous DICOM object(s); and
transmitting an analysis of at least one of the uncompressed and validated anonymous DICOM object(s), the analysis having been completed according to the priority level of the unique case file.

10. The system of claim 9, wherein the analysis is further comprised of:
generating a volume image file based on at least one of the anonymous DICOM object(s) of the unique case file, the volume image file representing a three dimensional (3D) volume;
generating an archive file including analysis data associated with the volume image file; and
generating a patient-specific model based on the volume image file and the archive file.

11. The system of claim 10, wherein the volume image file includes a file checksum that correlates the volume image file to the archive file.

12. The system of claim 9, wherein the system is further configured for:
monitoring a status of the unique case file; and
advancing analysis of the unique case file based on the status and the priority level of the unique case file.

13. The system of claim 9, wherein the system is further configured for:
setting the priority level for the unique case file based on an entity generating one or more DICOM object(s) associated with the one or more anonymous DICOM objects and/or an entity analyzing the one or more anonymous DICOM objects.

14. The system of claim 9, wherein the system is further configured for:
generating a draft result report based on the received analysis; and
generating an interactive user interface of the draft result report.

15. The system of claim 9, wherein the system is further configured for:
receiving a modification to the draft result report;
generating a final result report based on the modification; and
storing the final result report, a pre-defined identified network address, or a requesting web interface.

16. The system of claim 15, wherein the final result report is pushed to a database, a pre-defined identified network address, or a requesting web interface using secure Hypertext Transfer Protocol (HTTPS).

17. A non-transitory computer readable medium for use on a computer system containing computer-executable programming instructions for analyzing health data over a decentralized cloud-computing platform, the method comprising:
receiving a unique case file containing one or more anonymous DICOM object(s) for analysis;
setting a priority level for the unique case file based on priorities associated with the one or more anonymous DICOM object(s);
uncompressing and validating at least one of the one or more anonymous DICOM object(s); and
transmitting an analysis of at least one of the uncompressed and validated anonymous DICOM object(s), the analysis having been completed according to the priority level of the unique case file.

18. The non-transitory computer readable medium of claim 17, wherein the analysis comprises:
generating a volume image file based on at least one of the anonymous DICOM object(s) of the unique case file, the volume image file representing a three dimensional (3D) volume;
generating an archive file including analysis data associated with the volume image file; and
generating a patient-specific model based on the volume image file and the archive file.

19. The non-transitory computer readable medium of claim 17, the method further comprising:
monitoring a status of the unique case file; and
advancing analysis of the unique case file based on the status and the priority level of the unique case file.

20. The non-transitory computer readable medium of claim 17, the method further comprising:
setting the priority level for the unique case file based on an entity generating one or more DICOM object(s) associated with the one or more anonymous DICOM objects and/or an entity analyzing the one or more anonymous DICOM objects.
